(19) United States
(12) Patent Application Publication
(10) Pub. No.: US 2015/0284795 A1
(43) Pub. Date: Oct. 8, 2015

(54) Method for determining age independently of sex

(71) Applicants: Eugenia MAKRANTONAKI, (US); Christos C. ZOUBOULIS, Berlin (DE)

(72) Inventors: Eugenia Makranrantonaki, Wildeshausen (DE); Christos C. Zouboulis, Berlin (DE); James Adjaye, Dusseldorf (DE)

(21) Appl. No.: 14/439,639

(22) PCT Filed: Nov. 1, 2013
(86) PCT No.: PCT/EP2013/072878

§ 371 Date: Apr. 29, 2015

(30) Foreign Application Priority Data
Nov. 1, 2012 (DE) 10 2012 110 469.7

(51) Int. Cl. C12Q 1/68; G01N 33/68

(52) U.S. Cl. CPC C12Q 1/6876; G01N 33/6881; C12Q 2600/16; C12Q 2600/124

(57) ABSTRACT

The present invention relates to a method for the determination of the aging condition of a subject. The method allows conclusions on the age and age-related conditions of a subject—also regardless of gender-based on specific nucleic acids or proteins and the expression level thereof. The present invention relates further to a group of genes, which expression level is dependent from the aging condition of the respective subject—also regardless of gender. Furthermore, the present invention relates to the determination of the expression level of the genes, as well as to the use of the respective expression levels for the determination of aging conditions. This invention also relates to devices, particularly arrays and chips, which can be applied for the determination of the aging condition of a subject.

The present invention relates to a method for the determination of the aging condition of a subject. The method allows drawing conclusions on the age and age-related conditions of a subject—also regardless of the gender-, based on particular nucleic acids or proteins and the expression levels thereof. In the context of this invention, age-related conditions particularly can be age-associated diseases.

The present invention also relates to a group of genes the expression levels thereof—also regardless of the gender—being depended on the aging condition of the respective subject. Furthermore, the invention relates to the determination of the expression level of the genes and the use of the respective expression levels for the determination of aging conditions.

The invention also relates to devices, particularly arrays and chips, which can be used for the determination of the aging condition of a subject.

In today's opinion, aging is a complex multifactorial process that is subject to both hereditary and external influences. The exact mechanisms of aging are not known. However, there are several theories about aging mechanisms, which are not necessarily mutually exclusive, but can quite have an impact on the overall process of aging as parallel or sequential processes in terms of a multifactorial genesis. These theories include on the one hand changes of the genetic information and its structure, like, for example, a progressive shortening of the telomeres, chromosomal abnormalities and increased occurrence of mutations caused or accompanied by a reduced capacity for DNA repair. On the other hand, it is believed that oxidative stress, also associated with dysfunctions of mitochondria, and a higher incidence of misfolded proteins triggered by a reduction of the protein folding capacity and/or a reduction of the degradation of misfolded proteins, are associated with aging. However, it is still not known whether these observed effects are reason or consequence of aging.

Due to the increasing life expectancy it is to be expected that in the future age-related phenomena will be of increased importance to society. Therefore, there are made great worldwide efforts to determine more closely the influence of various factors on aging and to find possible aging-slowing influences and preventive measures for age-associated diseases, respectively. Thereby, a determination of the progression of aging and healthy aging, respectively, based on objective, well-defined and reproducibly determinable parameters is of central importance for the study of influences on aging. These parameters must differ in different aging conditions in at least one measurable property.

From WO 2010/104573 A1 already a method is known according to which particular genes are to be identified, which can be used for the determination of the age of a subject. The determination of the aging condition according to the method described therein, however, is very difficult as the samples on which the method is to be carried out are difficult to access and partially require surgical intervention. The determination of the aging condition according to the method described therein is therefore very difficult.

In the state of the art, no method is known according to which the aging condition of a subject can be determined easily and with a reasonable confidence. It is also not known from the state of the art, to what extend the expression levels of particular biomarkers show gender-specific differences. It is, however, of immense importance for the use of genes as biomarkers that a gender-independent determination of the aging condition of a subject is possible. This is not the case, if the respective gene is suitable as biomarker for one of the genders only. In the previously known biomarkers for aging the situation is even more unfavorable, as it is not even known whether they can be used gender-independently. As a result, misinterpretations of the collected data may occur.

It is therefore an object of the present invention to provide a method, which allows for a reliable and fast, also gender-independent, determination of the aging condition of a subject. In addition, it is an object of the present invention to allow for a reliable and fast, also gender-independent, determination of the healthy aging condition of a subject, so that individual variations, which may lead to diseases can be determined. The object is solved by a method comprising the following steps:
a. providing a cell sample of a subject,
b. determining the expression level of at least one gene in the cell sample, whose expression level in said cell sample is dependent on the aging condition of the subject,
c. comparing the expression level of the gene with at least one comparison value, wherein the comparison value is the expression level of the respective gene in a control sample and wherein the aging condition of the control sample is known.

As subjects whose cells are suitable for providing a cell sample, living beings, particularly animals, are possible. Preferred subjects are mammals, a preferred subject is man.

“Expression level” according to the invention refers to the content of gene product of a particular gene per cell. The gene product may be present both in the form of RNA or in the form of proteins. The determination of the expression level can therefore comprise determining the amount of a protein or nucleic acid, particularly RNA.

“Aging condition” according to the invention, preferably refers to the postnatal age of the subject. In cell samples from cell cultures, preferably the number of passages in the respective culture is regarded as the aging condition. The aging condition can also be the presence of an age-associated disease or another age-associated condition. Age-associated diseases particularly are cancerous diseases, however, also auto-immune diseases, e.g. bullous dermatoses, lupus erythematosus, infectious diseases, e.g. erysipelas, urinary tract infection, pneumonia, zoster, metabolic diseases, e.g. metabolic syndrome, vascular diseases, e.g. chronic venous insufficiency, arterial occlusive disease, neurodegenerative diseases, e.g. Alzheimer's disease, Parkinson's disease, musculoskeletal disorders, e.g. osteoporosis, diseases of internal organs, e.g. coronary heart disease, COPD, precancerosis and tumors of the skin, e.g. actinic keratoses, basal cell carcinoma, squamous cell carcinoma, leg ulcers (open leg).

The comparison value according to the present invention preferably is determined by determining the expression levels in cell samples of subjects having known aging condition. For example, the comparison value may be determined by examining a group of at least 2, preferably at least 5 and particular preferably at least 10 comparison subjects with regard to the expression level of the considered gene or the considered genes (control sample). Accordingly, the same steps a and b are preformed with the comparison subjects as for the subject, which is to be examined. The result returns the comparison value and allows a relative statement regarding the aging condition of the examined subject. It is apparent, that hereby a statement is possible with regard to almost any aging condition. The comparison subject or the comparison subjects, respectively, preferably belong to the same species, as the subject to be examined.

In a preferred embodiment the cell sample is a fixed animal tissue, particularly a skin sample, a saliva sample, a smear cell sample, a blood sample, a urine sample or a stool sample. In a preferred embodiment of the invention the cell sample comprises at least one homogenate of cells. The cell sample can be obtained subsequent to appropriate preparation, from cell- or tissue cultures or from tissues of living organisms, particularly from a skin sample, a saliva sample, a smear cell sample, a blood sample, a urine sample or a stool sample.

According to the present invention, both cells having a female and cells having a male genotype are useful as cell samples. According to the present invention, preparations of the skin, preparations of the mucous membrane, a smear cell sample, a blood sample or a urine sample can be used as cell samples. This is of particular advantage, as skin, oral, genital and anal mucous membrane, blood, urine and stool are easily accessible for sampling in general. In connection therewith, non-sun-exposed skin areas are particularly useful as cell sample, as the aging there is proceeding largely independent from external factors. Non-sun-exposed skin areas typically are the inner side of the upper arm or the inner side of the thigh, and the buttocks.

The aging condition of the skin allows conclusions about the aging condition of all other tissues being being subject to aging. Particularly, the aging condition of the skin allows conclusions on the aging condition of other tissues of ectodermal origin. Tissues of ectodermal origin in accordance with the present invention preferably are the central nervous system, the peripheral nervous system, teeth, hair and nails.

The aging condition of the skin, the mucous membrane, the blood, the urine and the stool allows conclusions on the aging condition of all other tissues being subject to aging.

In dead organisms cell samples can be used from any tissue for the determination of the aging condition.

The comparison of the expression level of the gene with at least one comparison value, wherein the comparison value is the expression level of the respective gene in a control sample, and wherein the aging condition of the control sample is known, is carried out to determine any matches or differences of the expression level measured in the cell sample and the expression level of the comparison value.

The present invention comprises a group of genes having an age-related change in expression, and their use as biomarker for aging and/or age-associated conditions. The determination of the expression level, thereby can be performed with the method known to the person skilled in the art, both at the RNA- or the protein-level.

Preferred methods for determining the expression levels are Northern blotting, RNase protection, real-time PCR, macro- and microarrays, SILAC, label-free absolute quantification, Western blotting, ELISA, immunocytochemistry and immunohistochemistry.

For the determination of the level of expression at the RNA level, preferably classic electrophoretic methods are to be used. These particularly are Northern blotting, RNase protection, more preferably PCR-based methods like real-time PCR. Still more preferably, hybridization procedures like macro- and microarrays are to be used. A particularly preferred method is the real-time PCR.

For the determination of the expression level at protein level, preferably mass spectrometric methods are to be used. These particularly are SILAC and label-free absolute quantification. Particularly preferred antibody-based methods like Western blotting, ELISA and immunocytochemistry and immunohistochemistry are to be used. A particularly preferred method is the immunohistochemistry.

A preferred application of the invention is the analysis of the effect of cosmetic and medicinal products on the aging condition, particularly the skin aging. For this purpose, for example, products slowing the aging can be applied to defined areas of the skin, and the effect can be compared using the analysis of the gene expression of the described genes on the treated and untreated sites. Predispositions for particular age-related diseases may also be identified.

A further preferred application of the invention is the analysis of the influence of medical products to the aging of particular tissues or the whole organism. For this purpose, for example, products slowing the aging of particular tissues or of the whole organism can be taken systemically and the effect can be compared using the analysis of the gene expression of the described genes in cell samples of the skin before and after the taking of the medical product. Predispositions for particular age-related diseases can also be identified.

A further preferred application is the analysis, for example, of traces fount at crime scenes, which allows for a restriction of the age of the offender and thus may restrict the number of possible suspects. It is known to the respective person skilled in the art that often skin scales or other tissue of the offender can be secured at the crime scene, which then may be used for a respective analysis in the purposes of the invention.

A further possible application is the analysis of cells from cell cultures. Particularly, possible age-promoting or age-slowing factors may be tested, for example, in high-throughput-screening-methods, by qualitatively and quantitatively comparing the gene expression level of the genes described in this application with the respective comparison values of untreated cell cultures under the influence of the respective factors.

The genes preferably are selected from the following human genes or homologs thereof:

RDH16 (Unigene ID Hs.134958, nucleotide sequence SEQ ID NO: 1, amino acid sequence SEQ ID NO: 2),

MGC3101 (Unigene ID Hs.301394, nucleotide sequence SEQ ID NO: 3, amino acid sequence SEQ ID NO: 4),

C9orf112 (Unigene ID Hs.292570, nucleotide sequence SEQ ID NO: 5, amino acid sequence SEQ ID NO: 6),

STK40 (Unigene ID Hs.471768, nucleotide sequence SEQ ID NO: 7, amino acid sequence SEQ ID NO: 8),

TOM1L2 (Unigene ID Hs.462379, nucleotide sequence SEQ ID NO: 9, amino acid sequence SEQ ID NO: 10),

GAMT (Unigene ID Hs.81131, nucleotide sequence SEQ ID NO: 11, amino acid sequence SEQ ID NO: 12),

MFSD3 (Unigene ID Hs.7678, nucleotide sequence SEQ ID NO: 13, amino acid sequence SEQ ID NO: 14),

C19orf24 (Unigene ID Hs.591383, nucleotide sequence SEQ ID NO: 15, amino acid sequence SEQ ID NO: 16),

TRIM33 (Unigene ID Hs.26837, nucleotide sequence SEQ ID NO: 17, amino acid sequence SEQ ID NO: 18),

LRIG3 (Unigene ID Hs.253736, nucleotide sequence SEQ ID NO: 19, amino acid sequence SEQ ID NO: 20),

DOCK9 (Unigene ID Hs.596105, nucleotide sequence SEQ ID NO: 21, amino acid sequence SEQ ID NO: 22),

NLGN2 (Unigene ID Hs.26229, nucleotide sequence SEQ ID NO: 23, amino acid sequence SEQ ID NO: 24),

B3GALT3 (Unigene ID Hs.418062, nucleotide sequence SEQ ID NO: 25, amino acid sequence SEQ ID NO: 26),

FZD7 (Unigene ID Hs.173859, nucleotide sequence SEQ ID NO: 27, amino acid sequence SEQ ID NO: 28),

TUBAL3 (Unigene ID Hs.163079, nucleotide sequence SEQ ID NO: 29, amino acid sequence SEQ ID NO: 30),

MMP27 (Unigene ID Hs.534479, nucleotide sequence SEQ ID NO: 31, amino acid sequence SEQ ID NO: 32),

CORIN (Unigene ID Hs.518618, nucleotide sequence SEQ ID NO: 33, amino acid sequence SEQ ID NO: 34),

PPARD (Unigene ID Hs.696032, nucleotide sequence SEQ ID NO: 35, amino acid sequence SEQ ID NO: 36),

SIRT6 (Unigene ID Hs.423756, nucleotide sequence SEQ ID NO: 37, amino acid sequence SEQ ID NO: 38),

CPT1B (Unigene ID Hs.439777, nucleotide sequence SEQ ID NO: 39, amino acid sequence SEQ ID NO: 40) and

MATN4 (Unigene ID Hs.278489, nucleotide sequence SEQ ID NO: 41, amino acid sequence SEQ ID NO: 42).

RDH16 encoding “retinol dehydrogenase 16”.

MGC3101 encoding “dysbindin domain-containing protein 1”.

C9orf112 encoding “WD repeat-containing protein 85”.

STK40 encoding “serine/threonine-protein kinase 40”.

TOM1L2 encoding “TOM1-like protein 2”.

GAMT encoding “guanidinoacetate N-methyltransferase”.

MFSD3 encoding “major facilitator superfamily domain-containing protein 3”.

C19orf24 encoding “uncharacterized membrane protein C19orf24”.

TRIM33 encoding “E3 ubiquitin-protein ligase TRIM33”.

LRIG3 encoding “leucine-rich repeats and immunoglobulin-like domains protein 3”.

DOCK9 encoding “dedicator of cytokinesis protein 9”.

NLGN2 encoding “neuroligin-2”.

B3GALT3 encoding “UDP-GalNAc:beta-1,3-N-acetylgalactosaminyltransferase 1”.

FZD7 encoding “frizzled-7”.

TUBAL3 encoding “tubulin alpha chain-like 3”.

MMP27 encoding “matrix metalloproteinase-27”.

CORIN encoding “atrial natriuretic peptide-converting enzyme”.

PPARD encoding “peroxisome proliferator-activated receptor delta”.

SIRT6 encoding “NAD-dependent protein deacetylase sirtuin-6”.

CPT1B encoding “Carnitine O-palmitoyltransferase 1B”.

MATN4 encoding “Matrilin-4”.

These genes have age-dependent expression levels, particularly in human skin cells. In the context of the method according to the present invention the expression levels of one or more genes can be analyzed. It is a particular advantage that the expression levels can be determined in skin samples.

The herein described genes are shown in the hereto attached sequence listing. In addition to the gene itself the respective proteins are also shown there. It is apparent that the sequences of the genes may be different due to naturally occurring polymorphisms in different individuals. Nevertheless, these genes which comprise polymorphisms can be used in the context of the present invention. Therefore, in the context of this description the genes set forth herein are also understood to be such genes which exhibit slight variations in the sequences. Also such genes are encompassed by the term chosen herein, which have a sequence identity with regard to the polynucleotide shown in the sequence listing of at least 95%, preferably of at least 96%, further preferably of at least 97%, more preferably of at least 98% and particularly preferably of at least 99%. Of course, also such embodiments, wherein the sequence identity is 100% are according to the invention. The sequence identity can be determined using the computer programs known to the person skilled in the art, for example, with computer programs from the Wisconsin Sequence Analysis Package, Version 8 (of Genetics Computer Group, Madison, USA), particularly BESTFIT, FASTA and GAP, which are based on the algorithm of Smith and Waterman. The programs can be applied with the default settings recommended by the manufacturer.

According to the present invention, the following genes are suitable: SIRT6, RDH16, CPT1B, MGC3101, C9orf112, STK40, TOM1L2, GAMT, MFSD3, C19orf24, TRIM33, LRIG3, DOCK9, NLGN2, B3GALT3, FZD7, TUBAL3, MMP27, MATN4, CORIN, PPARD and combinations thereof.

According to the present invention, the following genes are preferred: RDH16, MGC3101, C9orf112, STK40, TOM1L2, GAMT, MFSD3, C19orf24, TRIM33, LRIG3, DOCK9, NLGN2, B3GALT3, FZD7, TUBAL3, MMP27, CORIN, PPARD and combinations thereof.

According to the present invention, the following genes are also preferred: SIRT6, CPT1B, RDH16, STK40, GAMT, LRIG3, DOCK9, FZD7, MATN4, MMP27, CORIN, PPARD and combinations thereof.

In preferred embodiments, the expression levels of at least 2, further preferably at least 3, further preferably at least 4, further preferably at least 5, further preferably at least 6, further preferably at least 7, further preferably at least 8, further preferably at least 9, further preferably at least 10, further preferably at least 11, further preferably at least 12, further preferably at least 13, further preferably at least 14, further preferably at least 15, w further preferably at least 16, further preferably at least 17, further preferably at least 18, further preferably at least 19, further preferably at least 20 and particularly preferred 21 of the preferably examined genes are used according to the present invention. Even more than 21 genes can be used.

According to the present invention, the following genes are particularly preferably examined: C9orf112, STK40, TOM1L2, GAMT, MFSD3, C19orf24, TRIM33, LRIG3, DOCK9, NLGN2, B3GALT3 and FZD7. These genes gender-independently have age-dependently different expression levels, which is of particular advantage according to the present invention.

Further preferably, the genes are selected from C9orf112, TOM1L2, GAMT, MFSD3, C19orf24, TRIM33, LRIG3, DOCK9, NLGN2, B3GALT3 and FZD7. These genes show a qualitatively equal change of the expression level.

Particularly preferably the genes are selected from TOM1L2, NLGN2, B3GALT3 and FZD7. These genes show a particularly high age-dependent change of the expression level.

In a particularly preferred embodiment the expression level of FZD7 and/or PPARD is determined at the protein level.

In a further particularly preferred embodiment the expression level of CORIN, SIRT6, CPT1B and/or MATN4 is determined at the protein level.

In a further particularly preferred embodiment the expression level of CORIN, FZD7, PPARD, SIRT6, CPT1B and/or MATN4 is determined at the protein level.

In a preferred embodiment of the present application the method comprises the determination of an expression level of a gene selected from the group C9orf112, TOM1L2, GAMT, MFSD3, C19orf24, wherein an expression level, which is higher than the comparison value, means a stronger expressivity of the aging condition with regard to the comparison subject or the comparison subjects. The expression levels of these genes are gender-independently higher in individuals having a stronger expressivity of the aging condition. An expression level particularly is deemed to be higher as the comparison value when the expression level is at least 5%, further preferably at least 10%, particularly preferably at least 20% higher than the comparison value. Due to the advantageous properties of this method already such slight deviations can be robustly detected.

In a preferred embodiment of the present invention, the method comprises the determination of an expression level of a gene selected from the group TRIM33, LRIG3, DOCK9, NLGN2, B3GALT3, FZD7, wherein an expression level, which is lower than the comparison value, means a stronger expressivity of the aging condition with regard to the comparison subject or the comparison subjects. The expression levels of said genes are gender-independently lower. An expression level particularly is deemed to be lower as the comparison value when the expression level is at least 5%, further preferably at least 10%, particularly preferably at least 20% lower than the comparison value. Due to the advantageous properties of this method already such slight deviations can be robustly detected.

The present invention furthermore provides a device that can be used for the reliable and fast determination of the aging condition of a subject. The device preferably comprises a carrier material and biological molecules, which are immobilized on the carrier material at precisely defined positions. Particularly preferably, the device is a microarray and/or a biochip. A biochip according to the present invention can be both a DNA-chip and a protein chip.

The carrier material preferably consists of cardboard, paper, glass or plastic.

The biological molecules preferably comprise antibodies, which specifically recognize at least one epitope on at least one of the proteins encoded by the genes according to the present invention. Particularly preferably, the biological molecules comprise DNA-molecules having a linear sequence of at least 12 nucleotides, wherein the nucleotide sequence is selected from the sequence of one of the genes according to the present invention.

As a preferred embodiment, chips contain the preferred genes. They serve as target identification by genomics and proteomics, particularly in the form of biochips, of differentially expressed genes in age in both sexes.

In biochips, among others, specially coated plastic or coated glass is used as carrier material on which the tests, which are often only a few micrometers in size, are mounted (immobilized) with the help of machines. Frequently biochips are classified according to the substances that are determined in the test: In DNA-chips DNA- and RNA-fragments are detected, in protein-chips particular proteins are recognized—often with the help of antibodies.

The DNA chip-technology uses techniques from the semiconductor industry to identify known genes on a fingernail-sized plastic or glass plate, the microarray, and to measure their activity.

Aldehyde slides are preferred. In aldehyde slides aldehyde groups are attached to the surface of the chips, which bind the amino-modified oligonucleotides via a imine formation (covalent bond).

Further preferred are epoxy-modified slides. In epoxy-modified slides amino-modified oligonucleotides bind to the epoxide group to form an amine.

Further preferred are streptavidin-modified slides. In streptavidin-modified slides biotin-modified oligonucleotides bind to the protein. The tetramer streptavidin has four binding sites, each of which can bind a biotin.

Further preferred are NHS slides. These slides are coated with NHS-ester groups. By an amino-modified oligonucleotide an amide (NHS=N-hydroxysuccinimide) is formed.

Further preferred are amino slides. In amino slides an unmodified oligonucleotide binds to an indefinite position, during the irradiation with light in the UV range (indefinite reaction).

The mode of operation of the protein-chip-technology is comparable with an ELISA (Enzyme-linked Immunosorbent Assay) in a reduced format. The proteins are immobilized on a carrier chip (glass or plastic) in exact arrangement. Particularly preferred applied proteins are antibodies. Protein-chips, in which the used proteins are antibodies, are also referred to as antibody-chips. Basically, the protein-chip-technology provides the possibility to detect any binding to a protein in an assay-format. Further preferred coupling proteins are enzymes for the detection of particular substrates or antigens for the detection of particular antibodies in a biological sample.

A protein-chip is incubated with a biological sample and the binding of a biological marker to the immobilized protein is detected in the subsequent steps. The detection method varies depending on the experimental setup, preferably applied are immune assays in a displacement format.

Similar to a DNA-chip, compared to a Southern-blot or Northern-blot, the protein chip format provides several advantages over other techniques.
Reduction of the antibody and reagent consumption
Detection of biomarkers in lowest concentrations
integration of multiple assays on one chip (for example, various biomarkers)
miniaturization and automation of the analysis system for routine diagnosis

The present invention also provides detection kits with ready-to-use reagents, all-in-one-concepts—consisting of microarrays, hybridization station, scanner (e.g. fluorescence) and analysis software. An attractively priced alternative to the usual fluorescence scanners are systems which detect the hybridization electrochemically or via a silver precipitation on gold nanoparticles.

The present invention also provides computer systems with databases, where information about the expression of one or more polynucleotides according to the present invention, having a differential expression in young or elderly test persons, is collected.

The invention will be exemplarily illustrated on the following example. The example describes a preferred embodiment of the invention. The invention, however, is in any case not limited to an embodiment as described in the example.

### EXAMPLE 1

Skin samples of four groups of adult human subjects were taken. Thereby, the groups were as follows:

Group A: Young woman (age 26.7±4 years, n=7),

Group B: old women (age 70.75±3.3 years, n=4),

Group C: young men (age 25.8±5.2 years, n=6),

Group D: old men (age 76±3.8 years, n=7).

The expression of the genes described below was determined and averaged separately for each group. For determination of the expression subsequent to cell homogenization at first RNA was isolated and then transcribed into DNA by reverse transcription. This was then used as a basis for the preparation of biotinylated cRNA, which was in turn used for hybridization to human-8 BeadChips. The relative change of the expression level was determined as a ratio of the respective gene expressions of the older and the younger group. The ratios are given respectively as logarithm of the basis two (log 2).

| | NCBI | | Log₂ | P- | Log₂ | P- |
|---|---|---|---|---|---|---|
| Name | reference | description | (D/C) | value | (B/A) | value |
| SIRT6 | NM_016539.1 | Homo sapiens sirtuin | 1.246 | 0.030 | 1.762 | 0.027 |
| | | (silent mating type | | | | |
| | | information regulation 2 | | | | |
| | | homolog) 6 (S. | | | | |
| | | cerevisiae) (SIRT6), | | | | |
| | | mRNA. | | | | |
| RDH16 | NM_003708.2 | Homo sapiens retinol | 1.016 | 0.014 | 1.186 | 0.048 |
| | | dehydrogenase 16 (all- | | | | |
| | | trans and 13-cis) | | | | |
| | | (RDH16), mRNA. | | | | |
| CPT1B | NM_152246.1 | Homo sapiens carnitine | 1.000 | 0.013 | 1.021 | 0.007 |
| | | palmitoyltransferase 1B | | | | |
| | | (muscle) (CPT1B), | | | | |
| | | nuclear gene encoding | | | | |
| | | mitochondrial protein, | | | | |
| | | transcript variant 3, | | | | |
| | | mRNA. | | | | |
| MGC3101 | NM_024043.2 | Homo sapiens | 0.796 | 0.001 | 1.446 | 0.043 |
| | | hypothetical protein | | | | |
| | | MGC3101 (MGC3101), | | | | |
| | | mRNA. | | | | |
| C9orf112 | NM_138778.1 | Homo sapiens | 0.690 | 0.000 | 0.479 | 0.050 |
| | | chromosome 9 open | | | | |
| | | reading frame 112 | | | | |
| | | (C9orf112), mRNA. | | | | |
| STK40 | NM_032017.1 | Homo sapiens | 0.621 | 0.011 | −0.419 | 0.023 |
| | | serine/threonine kinase | | | | |
| | | 40 (STK40), mRNA. | | | | |
| TOM1L2 | NM_001033551.1 | Homo sapiens target of | 0.601 | 0.001 | 0.674 | 0.033 |
| | | myb1-like 2 (chicken) | | | | |
| | | (TOM1L2), transcript | | | | |
| | | variant 1, mRNA. | | | | |
| GAMT | NM_000156.4 | Homo sapiens | 0.496 | 0.003 | 0.738 | 0.042 |
| | | guanidinoacetate N- | | | | |
| | | methyltransferase | | | | |
| | | (GAMT), transcript | | | | |
| | | variant 1, mRNA. | | | | |
| MFSD3 | NM_138431.1 | Homo sapiens major | 0.452 | 0.028 | 0.595 | 0.034 |
| | | facilitator superfamily | | | | |
| | | domain containing 3 | | | | |
| | | (MFSD3), mRNA. | | | | |
| C19orf24 | NM_017914.2 | Homo sapiens | 0.395 | 0.049 | 0.627 | 0.043 |
| | | chromosome 19 open | | | | |
| | | reading frame 24 | | | | |
| | | (C19orf24), mRNA. | | | | |
| TRIM33 | NM_033020.2 | Homo sapiens tripartite | −0.396 | 0.002 | −0.515 | 0.048 |
| | | motif-containing 33 | | | | |
| | | (TRIM33), transcript | | | | |
| | | variant b, mRNA. | | | | |
| LRIG3 | NM_153377.3 | Homo sapiens leucine- | −0.398 | 0.002 | −0.650 | 0.029 |
| | | rich repeats and | | | | |
| | | immunoglobulin-like | | | | |
| | | domains 3 (LRIG3), | | | | |
| | | mRNA. | | | | |
| DOCK9 | NM_015296.1 | Homo sapiens dedicator | −0.415 | 0.014 | −0.815 | 0.009 |
| | | of cytokinesis 9 | | | | |
| | | (DOCK9), mRNA. | | | | |
| NLGN2 | NM_020795.2 | Homo sapiens neuroligin | −0.436 | 0.025 | −0.476 | 0.049 |
| | | 2 (NLGN2), mRNA. | | | | |
| B3GALT3 | NM_003781.2 | Homo sapiens UDP- | −0.590 | 0.043 | −0.575 | 0.043 |
| | | Gal:betaGlcNAc beta | | | | |
| | | 1,3- | | | | |
| | | galactosyltransferase, | | | | |
| | | polypeptide 3 | | | | |
| | | (B3GALT3), transcript | | | | |
| | | variant 1, mRNA. | | | | |
| FZD7 | NM_003507.1 | Homo sapiens frizzled | −0.915 | 0.045 | −0.910 | 0.026 |
| | | homolog 7 (Drosophila) | | | | |
| TUBAL3 | NM_024803.1 | Homo sapiens tubulin, | −0.915 | 0.029 | 0.673 | 0.043 |
| | | alpha-like 3 (TUBAL3), | | | | |
| | | mRNA. | | | | |
| MMP27 | NM_022122.2 | matrix metalloprotease | −0.982 | 0.048 | −1.579 | 0.001 |
| | | 27 | | | | |
| MATN4 | NM_003833.2 | Homo sapiens matrilin 4 | −1.393 | 0.014 | −1.823 | 0.003 |
| | | (MATN4), transcript | | | | |
| | | variant 1, mRNA. | | | | |
| CORIN | NM_006587.2 | Homo sapiens corin, | −1.935 | 0.035 | −3.216 | 0.014 |
| | | serine peptidase | | | | |
| | | (CORIN), mRNA. | | | | |

The example shows that the described genes gender-independently exhibit an age-dependent change of the expression level. In particular, it can be seen that the majority of genes have a qualitatively same age-dependent change in the expression level. The example also shows that depending on the gene, the age-dependent change in the expression level can mean an increase or a reduction of the expression level.

### EXAMPLE 2

The expression level of FZD7 and of PPARD in a human skin sample of women (n1=3) and men (n2=3) from non-sun-exposed areas was determined on the protein level using immunohistochemistry. In case of FZD7 a significantly higher expression level was detected in cell samples of younger test persons compared to cell samples of older test persons. In case of PPARD a significantly lower expression level was detected in cell samples of younger test persons compared to cell samples of older test persons.

Example 2 exemplarily shows that the determination of the age-dependent expression level of one of the genes according to the present invention may also be performed at the protein level. This is especially true for both genes, whose expression level is increased in old age, and genes, whose expression level is reduced in old age.

### EXAMPLE 3

The expression level of SIRT6, CPT1B, MATN4 and CORIN in human skin samples of women (n1=3) and men (n2=3) from non-sun-exposed areas were determined at the protein level using immunohistochemistry. In case of SIRT6 and CPT1B a significantly lower expression level was detected in cell samples of younger test persons in both sexes compared to cell samples of older test persons. In case of MATN4 and CORIN a significantly higher expression level was detected in cell samples of younger test persons compared to cell samples of older test persons in both sexes.

Example 3 exemplarily shows that the determination of the age-dependent expression level of one of the genes according to the present invention also can be performed at the protein level. This is especially true for both, genes whose expression level is increased in old age and genes whose expression level is reduced in old age

1. A method for the determination of the aging condition of a subject, comprising
a. providing a cell sample of the subject,
b. determining the expression level of at least one gene, whose expression level is dependent on the aging condition of the subject, and
c. comparing the expression level of the gene with at least one comparison value, wherein the comparison value is the expression level of the respective gene in a control sample.

2. The method according to claim 1, wherein the expression level is gender-independently dependent on the aging condition of the subject.

3. The method according to claim 1, wherein the gene is selected from one or more of the following human genes or a homologous gene thereof: CORIN (SEQ ID NO: 33), SIRT6 (SEQ ID NO: 37), CPT1B (SEQ ID NO: 39), MATN4 (SEQ ID NO: 41), RDH16 (SEQ ID NO: 1), MGC3101 (SEQ ID NO: 3), C9orf112 (SEQ ID NO: 5), STK40 (SEQ ID NO: 7), TOM1L2 (SEQ ID NO: 9), GAMT (SEQ ID NO: 11), MFSD3 (SEQ ID NO: 13), C19orf24 (SEQ ID NO: 15), TRIM33 (SEQ ID NO: 17), LRIG3 (SEQ ID NO: 19), DOCK9 (SEQ ID NO: 21), NLGN2 (SEQ ID NO: 23), B3GALT3 (SEQ ID NO: 25), FZD7 (SEQ ID NO: 27), TUBAL3 (SEQ ID NO: 29), MMP27 (SEQ ID NO: 31) and PPARD (SEQ ID NO: 35).

4. The method according to claim 1, wherein the gene is selected from one or more of the following human genes or a homologous gene thereof: CORIN (SEQ ID NO: 33), RDH16 (SEQ ID NO: 1), MGC3101 (SEQ ID NO: 3), C9orf112 (SEQ ID NO: 5), STK40 (SEQ ID NO: 7), TOM1L2 (SEQ ID NO: 9), GAMT (SEQ ID NO: 11), MFSD3 (SEQ ID NO: 13), C19orf24 (SEQ ID NO: 15), TRIM33 (SEQ ID NO: 17), LRIG3 (SEQ ID NO: 19), DOCK9 (SEQ ID NO: 21), NLGN2 (SEQ ID NO: 23), B3GALT3 (SEQ ID NO: 25), FZD7 (SEQ ID NO: 27), TUBAL3 (SEQ ID NO: 29), MMP27 (SEQ ID NO: 31) and PPARD (SEQ ID NO: 35).

5. The method according to claim 1, wherein the gene is selected from one or more of the following human genes or a homologous gene thereof: C9orf112 (SEQ ID NO: 5), STK40 (SEQ ID NO: 7), TOM1L2 (SEQ ID NO: 9), GAMT (SEQ ID NO: 11), MFSD3 (SEQ ID NO: 13), C19orf24 (SEQ ID NO: 15), TRIM33 (SEQ ID NO: 17), LRIG3 (SEQ ID NO: 19), DOCK9 (SEQ ID NO: 21), NLGN2 (SEQ ID NO: 23), B3GALT3 (SEQ ID NO: 25) and FZD7 (SEQ ID NO: 27).

6. The method according to claim 1, wherein the cell sample is a skin sample, a mucous membrane sample, a smear cell sample, a blood sample, a urine sample or a stool sample.

7. The method according to claim 1, wherein the cell sample is a sample from a dead organism.

8. The method according to claim 1, wherein the expression level is determined at an RNA-level or at a protein-level.

9. The method according to claim 1, wherein the subject is a mammal.

10. The method according to claim 1, wherein the cell sample comprises cells from cell-culture.

11. A device for the determination of the aging condition of a subject, particularly a chip or array, comprising
a. a carrier material
b. biological molecules being immobilized on the carrier material at precisely defined positions.

12. The device according to claim 11, wherein the biological molecules comprise DNA molecules having a linear sequence of at least 12 nucleotides, and the nucleotide sequence is selected from the sequence of one of said genes in claim 3.

13. The device according to claim 10, wherein the biological molecules comprise DNA molecules having a linear sequence of at least 12 nucleotides, and the nucleotide sequence is selected from the sequence of one of said genes in claim 4 or 5.

14. The device according to claim 10, wherein the biological molecules are antibodies, which specifically recognize at least one epitope on at least one of the proteins encoded by said genes in claim 3.

15. The device according to claim 10, wherein the biological molecules are antibodies, which specifically recognize at least one epitope on at least one of the proteins encoded by one of said genes in claim 4.
